(19) United States
(12) Patent Application Publication
(10) Pub. No.: US 2025/0084026 A1
(43) Pub. Date: Mar. 13, 2025

(54) NOVEL SALT OF 1-SULFONYL PYRROLE DERIVATIVE, PREPARATION METHOD THEREOF AND PHARMACEUTICAL COMPOSITION COMPRISING THEREOF

(71) Applicant: ILDONG PHARMACEUTICAL CO., LTD., Seoul (KR)

(72) Inventors: Hong Chul Yoon, Gyeonggi-do (KR); Joon Tae Park, Gyeonggi-do (KR); Jung Woo Lee, Gyeonggi-do (KR); Kyung Mi An, Gyeonggi-do (KR); Chang Hee Hong, Gyeonggi-do (KR); Jae Eui Shin, Gyeonggi-do (KR); Soo Jin Lee, Gyeonggi-do (KR); Hanna Seo, Gyeonggi-do (KR); Jae Hong Lee, Gyeonggi-do (KR)

(21) Appl. No.: 18/720,436

(22) PCT Filed: Dec. 14, 2022
(86) PCT No.: PCT/KR2022/020324

§ 371 Date: Jun. 14, 2024

(30) Foreign Application Priority Data
Dec. 15, 2021 (KR) 10-2021-0180017

(51) Int. Cl. C07C 57/15; A61K 31/194; C07C 51/42; C07D 401/12

(52) U.S. Cl. CPC C07C 57/15; A61K 31/194; C07C 51/42; C07D 401/12; C07B 2200/13

(57) ABSTRACT

The present disclosure relates to a novel salt of a 1-sulfonyl pyrrole derivative, and to a novel salt having excellent solubility in vivo, stability, bioavailability, and the like, a preparation method thereof, and a pharmaceutical composition comprising the same.

### TECHNICAL FIELD

The present disclosure relates to a novel salt of 1-sulfonyl pyrrole derivative, which is difficult to be prepared by a conventional salt preparation process, a preparation method thereof, and a pharmaceutical composition comprising the same.

### BACKGROUND ART

Potassium Competitive Acid Blockers (P-CABs) class anti-ulcer drugs inhibit gastric acid secretion by competitively binding to the K⁺ binding site of the proton pump, which is located in the final stage of acid secretion in gastric parietal cells, to interfere with the exchange of protons (H+). The P-CABs are next-generation drugs that compensate for the shortcomings of proton pump inhibitor (PPI) drugs such as omeprazole, esomeprazole, and ilaprazole, which are commonly prescribed in the gastric acid secretion inhibitor market at present.

These potassium-competitive gastric acid secretion inhibitors are in the spotlight as a new class of drugs, but the number of related drugs available on the market is still quite limited.

Meanwhile, many free bases may have problems in some cases in that they exist in the form of oil at room temperature of 15-25° C., or it is not easy to handle industrially.

Therefore, there is a need for research on a method for providing a pharmaceutical product that is advantageous in product handling and storage due to superior physical and chemical stability, non-hygroscopicity, and the like, achieves mass-production, and has excellent solubility, resulting in providing improved bioavailability. In particular, in order for a drug to exhibit rapid pharmacological activity in vivo, the drug should rapidly elute from the digestive tract. This is closely related to the drug solubility, that is, the higher the solubility of the drug, the higher the drug dissolution rate and the rate of absorption in the digestive tract, and the higher the rate of absorption in the digestive tract, the faster and more effective blood concentration is achieved even at low doses, and thus it is possible to expect high efficacy and bioavailability of the drug.

It is therefore also required to select the optimal salt form. Under this background, the present inventors repeatedly researched, found that a fumarate salt of 1-(5-(2-fluorophenyl)-4-methoxy-1-((6-methoxypyridin-3-yl) sulfonyl)-1H-pyrrol-3-yl)-N-methylmethanamine had excellent stability, solubility and bioavailability and high purity compared to other commonly used pharmaceutically acceptable salt compounds, and further made a lot of research to achieve mass-production and completed the present disclosure.

### DISCLOSURE OF INVENTION

### Technical Problem

An object of the present disclosure is to provide a novel salt of 1-(5-(2-fluorophenyl)-4-methoxy-1-((6-methoxypyridin-3-yl) sulfonyl)-1H-pyrrole-3-yl)-N-methylmethanamine. In particular, another object of the present disclosure is to provide a novel salt with improved physicochemical and/or pharmaceutical properties such as solubility (specifically, solubility in vivo), stability (dissolution stability, storage stability, and the like), and the like; a preparation method thereof, and a pharmaceutical composition comprising the same.

### Solution to Problem

In one general aspect, there is provided a novel salt of 1-(5-(2-fluorophenyl)-4-methoxy-1-((6-methoxypyridin-3-yl) sulfonyl)-1H-pyrrole-3-yl)-N-methylmethanamine, a preparation method thereof, and a pharmaceutical composition comprising the same as an active ingredient.

Hereinafter, each detailed description is provided below.

### Novel salt of 1-(5-(2-fluorophenyl)-4-methoxy-1-((6-methoxypyridin-3-yl) sulfonyl)-1H-pyrrole-3-yl)-N-methylmethanamine

In order to achieve the above object, the present disclosure provides a fumarate salt of 1-(5-(2-fluorophenyl)-4-methoxy-1-((6-methoxypyridin-3-yl) sulfonyl)-1H-pyrrole-3-yl)-N-methylmethanamine.

The fumarate salt of 1-(5-(2-fluorophenyl)-4-methoxy-1-((6-methoxypyridin-3-yl) sulfonyl)-1H-pyrrole-3-yl)-N-methylmethanamine may be represented by the following Chemical Formula I:

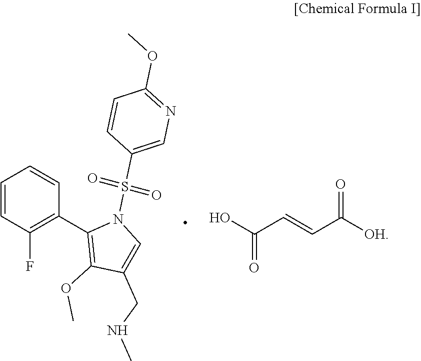 [SMILES: CNCc1cn(S(=O)(=O)c2ccc(OC)nc2)c(-c2ccccc2F)c1OC ; O=C(O)/C=C/C(=O)O]

The novel salt according to the present disclosure exhibits excellent physicochemical properties in various aspects such as stability, solubility in vivo, and bioavailability, and the like.

The fumarate salt of 1-(5-(2-fluorophenyl)-4-methoxy-1-((6-methoxypyridin-3-yl) sulfonyl)-1H-pyrrole-3-yl)-N-methylmethanamine of the present disclosure may have a thermogravimetric analysis (TGA) pattern showing a weight loss of less than 0.1 wt % at 120° C. or less. Specifically, the thermogravimetric analysis (TGA) pattern of FIG. 1 or FIG. 5 may be shown.

The fumarate salt (specifically, crystalline form thereof) of the present disclosure is characterized by having an endothermic transition peak value at 163 to 175° C. when a temperature increase rate is 20° C./min in a differential scanning calorimetry (DSC) graph, and preferably, is characterized by having an endothermic transition peak value at 165 to 173° C., and more preferably at 169±2° C.

Further, the fumarate salt of 1-(5-(2-fluorophenyl)-4-methoxy-1-((6-methoxypyridin-3-yl) sulfonyl)-1H-pyrrol-3-yl)-N-methylmethanamine may show the differential scanning calorimetry of FIG. 2 or FIG. 6.

Furthermore, the novel salt of the present disclosure is preferably in a crystalline form.

The crystalline form of the fumarate salt of the present disclosure may comprise, in an X-ray powder diffraction (XRPD) graph, at least three diffraction peaks at 2-theta (2θ) angle values selected from the group consisting of 12.87±0.2, 17.38±0.2, 18.55±0.2, 19.78±0.2, 22.62±0.2, 23.32±0.2, and 28.27±0.2. More specifically, the crystalline form of the fumarate salt of the present disclosure may comprise, in the XRPD graph, diffraction peaks at 2-theta (2θ) angle values of 12.87±0.2, 17.38±0.2, 18.55±0.2, 19.78±0.2, 22.62±0.2, 23.32±0.2, and 28.27±0.2.

More specifically, the crystalline form of the fumarate salt of the present disclosure may further comprise, in an X-ray powder diffraction (XRPD) graph, any one or more diffraction peaks at 2-theta (2θ) angle values selected from the group consisting of 14.32±0.2, 20.67±0.2, 21.74±0.2, and 25.95±0.2.

Further, the fumarate salt of 1-(5-(2-fluorophenyl)-4-methoxy-1-((6-methoxypyridin-3-yl) sulfonyl)-1H-pyrrol-3-yl)-N-methylmethanamine of the present disclosure may exhibit the X-ray powder diffraction spectroscopy pattern of FIG. 3 or FIG. 7.

In the present disclosure, a novel salt that had never been used in the related art was prepared. Specifically, a fumarate salt which is a novel salt of 1-(5-(2-fluorophenyl)-4-methoxy-1-((6-methoxypyridin-3-yl) sulfonyl)-1H-pyrrol-3-yl)-N-methylmethanamine may have excellent stability, photostability, thermal stability, and stability according to pH, thereby being stably maintained without a change in amount over a long period of time. In particular, the fumarate salt has excellent thermal stability against high temperature, and the like. Therefore, the raw material of the novel salt of the present disclosure is able to be obtained in high yield and high purity, and the increase in related substances is remarkably low even when stored for a long time, and thus high purity may be maintained for a long period of time.

In addition, the novel salt of the present disclosure may achieve excellent pharmacological effects by exhibiting excellent solubility values under various pH conditions, particularly bio-relevant media conditions, and may be usefully employed as a new active ingredient of a pharmaceutical composition capable of treating various indications.

According to an embodiment of the present disclosure, when preparing stimulated gastric fluid (SGF), fasted state simulated intestinal fluid (FaSSIF), and fed state simulated intestinal fluid (FcSSIF) and performing a test for measuring solubility and dissolution under conditions close to the in vivo environment, good solubility was shown. In particular, the solubility in FaSSIF was very good in the fumarate salt. It was confirmed from this finding that the novel salt according to the present disclosure had significantly excellent solubility in vivo and exhibited high bioavailability.

In addition, since the novel salt exhibits high bioavailability when administered orally, it is possible to exhibit excellent therapeutic effects even when taken in a small amount, thereby significantly improving the patient's medication compliance.

In addition, the novel salt of the present disclosure may have a rapid onset of action and a thermodynamically stable form, and may be very advantageous in processing and storage of pharmaceutical products to achieve easy formulation, and further, may maintain the same state even after the formulation is prepared so that the uniformity in view of the formulation amount may be stably maintained for a long period of time, and thus the novel salt may be easily applied to mass production.

### Preparation Method of Novel Salt of 1-(5-(2-fluorophenyl)-4-methoxy-1-((6-methoxypyridin-3-yl) sulfonyl)-1H-pyrrole-3-yl)-N-methylmethanamine

There is provided a preparation method of a fumarate salt of 1-(5-(2-fluorophenyl)-4-methoxy-1-((6-methoxypyridin-3-yl) sulfonyl)-1H-pyrrole-3-yl)-N-methylmethanamine represented by the following Chemical Formula I:

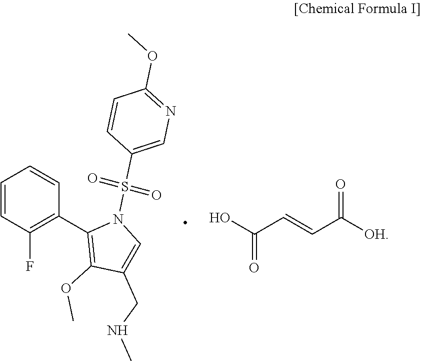 [SMILES: CNCc1cn(S(=O)(=O)c2ccc(OC)nc2)c(-c2ccccc2F)c1OC ; O=C(O)/C=C/C(=O)O]

Specifically, the preparation method of the present disclosure includes:
(1) dissolving a compound represented by the following Chemical Formula (II) in a single organic solvent or a mixed solvent to react with fumaric acid;

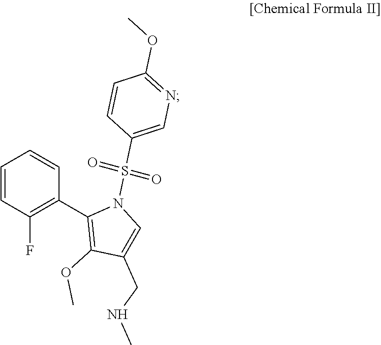 [SMILES: CNCc1cn(S(=O)(=O)c2ccc(OC)nc2)c(-c2ccccc2F)c1OC]

(2) precipitating a product from a reaction solution obtained in step (1); and
(3) filtering and drying the product of step (2).

In the preparation method of the present disclosure, the single organic solvent is preferably one selected from the group consisting of methanol, ethanol, isopropyl alcohol, normal propanol, acetone, methyl ethyl ketone, methyl acetate, ethyl acetate, tetrahydrofuran, 2-methyl tetrahydrofuran and acetonitrile. Isopropyl alcohol or acetone is more preferable. When isopropyl alcohol or acetone is used, the crystalline form of the fumarate salt has advantages of being stable and being able to be prepared in high yield and high purity.

In the preparation method of the present disclosure, the mixed solvent is a mixed solvent of (a) at least any one solvent selected from the group consisting of methanol, ethanol, 2-propanol, normal propanol, acetone, methyl ethyl ketone, acetonitrile, methyl acetate, ethyl acetate, tetrahydrofuran, and 2-methyl tetrahydrofuran, and (b) at least any one solvent selected from the group consisting of water, normal heptane, normal hexane, and diisopropyl ether.

A mixing ratio of the mixed solvent may be 1:1 to 1:20 by volume.

In the preparation method of the present disclosure, preferably, the step (1) may be performed at a temperature of 20 to 40° C., preferably at room temperature.

The fumaric acid in step (1) is preferably used in an amount of 0.5 to 2.0 equivalents, and more preferably in an amount of 0.7 to 1.3 equivalents, based on 1.0 equivalent of 1-(5-(2-fluorophenyl)-4-methoxy-1-((6-methoxypyridin-3-yl) sulfonyl)-1H-pyrrole-3-yl)-N-methylmethanamine.

In this step, the reaction may be performed for approximately 12 to 36 hours, preferably 20 to 30 hours, and more preferably 24 hours.

In step (2), the mixture may be cooled to a temperature of 0 to 10° C. or dried under low pressure conditions.

Then, after cooling or drying in step (2), the resulting product may be dried at a temperature of 20 to 70° C. or evaporated under nitrogen stream in step (3). Through the above process, it is possible to effectively remove residual solvents and the like and to obtain the desired crystalline form of the salt in high yield and high purity.

### Pharmaceutical Composition

The present disclosure provides a pharmaceutical composition comprising a fumarate salt of 1-(5-(2-fluorophenyl)-4-methoxy-1-((6-methoxypyridin-3-yl) sulfonyl)-1H-pyrrole-3-yl)-N-methylmethanamine represented by the following Chemical Formula I:

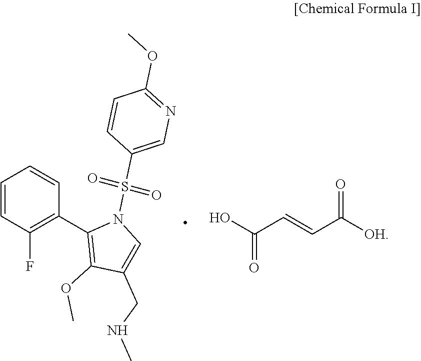 [SMILES: CNCc1cn(S(=O)(=O)c2ccc(OC)nc2)c(-c2ccccc2F)c1OC ; O=C(O)/C=C/C(=O)O]

The novel salt according to the present disclosure may have excellent stability, photostability, thermal stability, and pH stability, and may exhibit excellent solubility in vivo under bio-relevant media conditions close to the in vivo environment, thereby showing excellent pharmacological effects.

Accordingly, the pharmaceutical composition of the present disclosure is able to be used for the prevention or treatment of gastrointestinal ulcers, gastrointestinal inflammatory diseases or gastric acid-related diseases.

The gastrointestinal ulcer refers to an ulcer occurring in the digestive organs including both the stomach and intestines. Examples of the gastrointestinal ulcer may include, but are not limited to, peptic ulcer, gastric ulcer, duodenal ulcer, NSAID-induced ulcer, acute stress ulcer, Zollinger-Ellison syndrome, and the like. If the ulcer becomes serious, cancer may be developed. For example, in the case of the gastric ulcer, as the degree of the of the gastric ulcer becomes severe, the gastric ulcer may develop into gastric cancer.

In particular, the gastrointestinal ulcer may include damage to the gastric mucosa or damage to the small intestinal mucosa caused by drugs, alcohol, or the like. In particular, it may be damage to the gastric mucosa or damage to the small intestinal mucosa caused by NSAIDs or alcohol.

The gastrointestinal inflammatory disease refers to a disease caused by inflammation of the gastrointestinal tract.

Examples of the gastrointestinal inflammatory disease may include, but are not limited to, Helicobacter pylori infection, gastritis (for example, acute hemorrhagic gastritis, chronic superficial gastritis, chronic atrophic gastritis), inflammatory bowel disease, gastric MALT lymphoma, and the like.

The gastric acid-related disease refers to a disease caused by excessive secretion of gastric acid. Examples of the gastric acid-related disease may include, but are not limited to, erosive esophagitis, non-erosive esophagitis, reflux esophagitis, symptomatic gastroesophageal reflux disease (symptomatic GERD), functional dyspepsia, hyperacidity, upper gastrointestinal bleeding due to invasive stress, and the like.

According to the present disclosure, the gastrointestinal ulcer, gastrointestinal inflammatory disease or gastric acid-related disease may be any one or more selected from the group consisting of peptic ulcer, gastric ulcer, duodenal ulcer, NSAID-induced ulcer, acute stress ulcer, Zollinger-Ellison syndrome, Helicobacter pylori infection, gastritis, erosive esophagitis, non-erosive esophagitis, reflux esophagitis, inflammatory bowel disease, symptomatic gastroesophageal reflux disease (symptomatic GERD), functional dyspepsia, gastric cancer, gastric MALT lymphoma, hyperacidity, and upper gastrointestinal bleeding due to invasive stress.

The present disclosure provides a pharmaceutical composition comprising a fumarate salt of 1-(5-(2-fluorophenyl)-4-methoxy-1-((6-methoxypyridin-3-yl) sulfonyl)-1H-pyrrole-3-yl)-N-methylmethanamine represented by the following Chemical Formula I; and a pharmaceutically acceptable carrier:

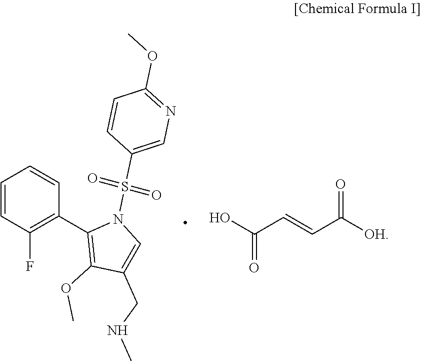 [SMILES: CNCc1cn(S(=O)(=O)c2ccc(OC)nc2)c(-c2ccccc2F)c1OC ; O=C(O)/C=C/C(=O)O]

In the present disclosure, the “pharmaceutically acceptable carrier” includes any and all solvents, dispersion media, coatings, antibacterial and antifungal agents, isotonic preparations, and absorption delaying agents, and the like that are physiologically compatible.

The composition of the present disclosure may be in various forms. These forms include, for example, liquid, semi-solid, and solid dosage forms such as liquid solutions (for example, injectable and infusible solutions), dispersions or suspensions, tablets, pills, powders, liposomes and suppositories. The form depends on the intended mode of administration and therapeutic use.

A typical composition is in the form of compositions similar to injectable and infusible solutions. One mode of administration is parenteral (for example, intravenous, subcutaneous, intraperitoneal, intramuscular).

For oral administration, the solid dosage forms may be provided as, for example, hard or soft capsules, pills, cachets, lozenges or tablets, each containing a predetermined amount of one or more compounds of the present disclosure. In still another embodiment, the oral administration may be provided in a powder or granular form.

In still another embodiment, the oral administration may be in a liquid dosage form. The liquid dosage form for oral administration includes, for example, pharmaceutically acceptable emulsions, solutions, suspensions, syrups, and elixirs containing an inert diluent (for example, water) commonly used in the art.

In still another embodiment, the present disclosure encompasses a parenteral dosage form. The “parenteral administration” includes, for example, subcutaneous injection, intravenous injection, intraperitoneal injection, intramuscular injection, intrasternal injection, and infusion. The injectable preparation (i.e., sterile injectable aqueous or oleaginous suspension) may be formulated according to known techniques using suitable dispersants, wetting and/or suspending agents.

Other carrier materials and the administration modes known in the pharmaceutical arts may also be employed. The pharmaceutical composition of the present disclosure may be prepared by any of the well-known pharmaceutical techniques, such as effective formulation and administration procedures.

These formulations may be prepared by conventional methods used for formulation in the art or a method disclosed in the document [see, Remington's Pharmaceutical Science (latest edition), Mack Publishing Company, Easton PA], and may be formulated into various formulations depending on each disease or component.

The composition of the present disclosure may be administered orally or parenterally (for example, intravenous, subcutaneous, intraperitoneal or topical application) according to the desired method, and the dose varies depending on the patient's weight, age, sex, health condition, diet, administration time, administration method, excretion rate, and severity of the disease. The daily dose of the novel salt of the present disclosure is about 0.01 to 500 mg/kg, preferably 1 to 100 mg/kg, and may be divided and administered once or several times a day.

The pharmaceutical composition of the present disclosure may further contain at least one active ingredient exhibiting the same or similar medicinal effect in addition to the novel salt.

### Therapeutic Use and Method for Treatment of Gastrointestinal Ulcers, Gastrointestinal Inflammatory Diseases or Gastric Acid-Related Diseases, and Use Thereof in the Manufacture of a Medicament for Treatment

The present disclosure provides a pharmaceutical composition for the prevention or treatment of gastrointestinal ulcers, gastrointestinal inflammatory diseases or gastric acid-related diseases, comprising: a fumarate salt of 1-(5-(2-fluorophenyl)-4-methoxy-1-((6-methoxypyridin-3-yl) sulfonyl)-1H-pyrrole-3-yl)-N-methylmethanamine represented by the following Chemical Formula I:

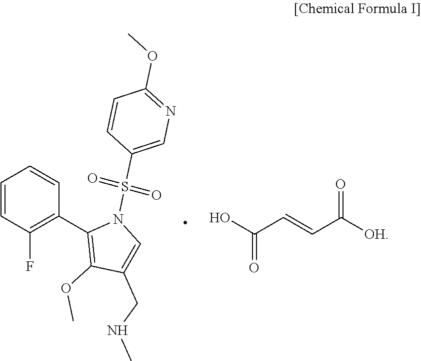 [SMILES: CNCc1cn(S(=O)(=O)c2ccc(OC)nc2)c(-c2ccccc2F)c1OC ; O=C(O)/C=C/C(=O)O]

The present disclosure provides a method for treating gastrointestinal ulcers, gastrointestinal inflammatory diseases or gastric acid-related diseases, comprising: administering to a subject in need thereof a therapeutically effective amount of the fumarate salt of 1-(5-(2-fluorophenyl)-4-methoxy-1-((6-methoxypyridin-3-yl) sulfonyl)-1H-pyrrole-3-yl)-N-methylmethanamine as described above.

The phrase “a subject in need thereof” means mammals including a human, which includes mammals such as humans, monkeys, cattle, horses, dogs, cats, rabbits, rats, and mice.

As used herein, the term “therapeutically effective amount” refers to an amount of the novel salt effective for the prevention or treatment of gastrointestinal ulcers, gastrointestinal inflammatory diseases, or gastric acid-related diseases, or a pharmaceutical composition comprising the same, and for example, may include, as an amount of the novel salt to be administered to the subject to be treated, any amount of the pharmaceutical composition comprising the above-described salts, to prevent occurrence or recurrence of gastrointestinal ulcers, gastrointestinal inflammatory diseases, or gastric acid-related diseases, to alleviate symptoms, to inhibit direct or indirect pathological consequences, to prevent metastasis, to reduce the rate of progression, or to alleviate or temporarily ameliorate the condition or to improve the prognosis. In other words, the therapeutically effective amount may be interpreted to encompass all doses in which symptoms of gastrointestinal ulcers, gastrointestinal inflammatory diseases, or gastric acid-related disease are improved or cured by the pharmaceutical composition.

The method for preventing or treating gastrointestinal ulcers, gastrointestinal inflammatory disease, or gastric acid-related diseases of the present disclosure includes not only treating the diseases themselves before the onset of signs, but also inhibiting or avoiding signs thereof by administering the above-described salt or the pharmaceutical composition comprising the same. In the management of diseases, a prophylactic or therapeutic dose of a particular active ingredient will vary depending on the nature and severity of the disease or condition and the route by which the active ingredient is administered. The dose and frequency of dose will vary depending on the age, weight and response of individual patients. A suitable dosage regimen may be readily selected by one of ordinary skill in the art considering these factors naturally. In addition, the method for treating gastrointestinal ulcers, gastrointestinal inflammatory diseases or gastric acid-related diseases using the pharmaceutical composition of the present disclosure may further include administering a therapeutically effective amount of an additional active agent useful for treating the diseases together with the above-described salt, wherein the additional active agent may exhibit synergistic or auxiliary effects with the above-described salt which is an active ingredient according to the present disclosure.

The present disclosure also provides use of a fumarate salt of 1-(5-(2-fluorophenyl)-4-methoxy-1-((6-methoxypyridin-3-yl) sulfonyl)-1H-pyrrole-3-yl)-N-methylmethanamine for the manufacture of a medicament for treatment of gastrointestinal ulcers, gastrointestinal inflammatory diseases or gastric acid-related diseases. The above-described salts for the manufacture of a medicament may be mixed with acceptable adjuvants, diluents, carriers, and the like, and may be prepared as a combined preparation with other active agents to have a synergistic action of the active ingredients.

Further, the present disclosure provides a pharmaceutical composition for the prevention or treatment of gastrointestinal ulcers, gastrointestinal inflammatory diseases or gastric acid-related diseases, comprising: a fumarate salt of 1-(5-(2-fluorophenyl)-4-methoxy-1-((6-methoxypyridin-3-yl) sulfonyl)-1H-pyrrole-3-yl)-N-methylmethanamine.

Matters mentioned in the uses, compositions and treatment methods of the present disclosure are applied equally as long as they do not contradict each other.

### Advantageous Effects of Invention

The novel salt according to the present disclosure may have excellent stability to increase the stability of the formulation and may have improved solubility (particularly, solubility in vivo) and bioavailability to be usefully employed as an active ingredient of a pharmaceutical composition.

### BRIEF DESCRIPTION OF DRAWINGS

The following drawings attached to the present specification illustrate preferred embodiments of the present disclosure, and serve to further understand the technical idea of the present disclosure together with the above-described content of the invention, and thus the present disclosure should not be construed as being limited only to the matters described in these drawings.

FIG. 1 is a thermogravimetric analysis (TGA) pattern analysis graph of a fumarate salt compound represented by Chemical Formula I prepared in Example 1 of the present disclosure.

FIG. 2 is a differential scanning calorimetry (DSC) pattern analysis graph of the fumarate salt compound represented by Chemical Formula I prepared in Example 1 of the present disclosure.

FIG. 3 is an X-ray powder diffraction analysis (XRPD) graph of the fumarate salt compound represented by Chemical Formula I prepared in Example 1 of the present disclosure.

FIG. 4 shows ¹H-NMR results for confirming ratios of cations and anions of the fumarate salt compound represented by Chemical Formula I prepared in Example 1 of the present disclosure.

FIG. 5 is a thermogravimetric analysis (TGA) pattern analysis graph of a fumarate salt compound represented by Chemical Formula I prepared in Example 3 of the present disclosure.

FIG. 6 is a differential scanning calorimetry (DSC) pattern analysis graph of the fumarate salt compound represented by Chemical Formula I prepared in Example 3 of the present disclosure.

FIG. 7 is an X-ray powder diffraction analysis (XRPD) graph of the fumarate salt compound represented by Chemical Formula I prepared in Example 3 of the present disclosure.

FIG. 8 shows ¹H-NMR results for confirming ratios of cations and anions of the fumarate salt compound represented by Chemical Formula I prepared in Example 3 of the present disclosure.

FIGS. 9A and 9B shows the results of dynamic vapor sorption (DVS) measurement of the fumarate salt compound represented by Chemical Formula I prepared in Example 3 of the present disclosure.

FIG. 10 is an XRPD graph of a phosphate salt of 1-(5-(2-fluorophenyl)-4-methoxy-1-((6-methoxypyridin-3-yl) sulfonyl)-1H-pyrrole-3-yl)-N-methylmethanamine prepared in Comparative Example 1.

FIG. 11 is a differential scanning calorimetry (DSC) pattern analysis graph of the phosphate salt of 1-(5-(2-fluorophenyl)-4-methoxy-1-((6-methoxypyridin-3-yl) sulfonyl)-1H-pyrrole-3-yl)-N-methylmethanamine prepared in Comparative Example 1.

FIG. 12 is a thermogravimetric analysis (TGA) pattern analysis graph of the phosphate salt of 1-(5-(2-fluorophenyl)-4-methoxy-1-((6-methoxypyridin-3-yl) sulfonyl)-1H-pyrrole-3-yl)-N-methylmethanamine prepared in Comparative Example 1.

### BEST MODE FOR CARRYING OUT THE INVENTION

Hereinafter, Examples and the like will be described in detail to assist the under-standing of the present disclosure. However, these Examples according to the present disclosure may be modified in various other forms, and the scope of the present disclosure should not be construed as being limited to the following Examples. These Examples of the present disclosure are provided to more fully explain the present disclosure to those of ordinary skill in the art.

### Instrument Used, Samples and Measurement Conditions

### 1) XRPD (X-Ray Powder Diffractometer)

The X-ray powder diffraction (XRPD) pattern was measured using XRD-6000 instrument manufactured by Shimadzu Corporation, and the conditions of use were set as shown in Table 1 below.

TABLE 1

| Settings | Parameters |
|---|---|
| Tube: | Cu: K-Alpha (λ = 1.54056A) |
| Generator: | Voltage: 40 KV Current: 30 mA |
| Scan Scope: | 2-50° (degree) |
| Scan Scope: | 5°/min |

### 2) DSC (Differential Scanning Calorimeter)

Using differential scanning calorimeter (DSC3) manufactured by Mettler Toledo, a compound sample (about 1 mg) was tested in a pinhole aluminum pan under nitrogen purge at a ramp rate of 20° C./min over the range of 30 to 300° C.

Specific conditions were set as shown in Table 2 below.

TABLE 2

| Settings | Parameters |
|---|---|
| Ramp rate | 20° C./min, over the range 30° C.~300° C. |
| Nitrogen purge | 50 mL/min |
| Samples weight | ~1 mg |

### 3) TGA (Thermal Gravimetric Analysis)

Using Pyris 1 TGA manufactured by PerkinElmer, Inc., a compound sample (about 5 mg) was weighed in pans under nitrogen purge at a ramp rate of 20° C./min over the range of 30 to 300° C.

Specific conditions were set as shown in Table 3 below.

TABLE 3

| Settings | Parameters |
|---|---|
| Ramp rate | 20° C./min, over the range 30° C.~300° C. |
| Nitrogen purge | 50 mL/min |
| Samples weight | ~5 mg |

### 4) ¹H-NMR (Nuclear Magnetic Resonance)

About 3 mg of the compound was weighed into a nuclear magnetic tube and 0.5 mL deuterated dimethyl sulfoxide was added to completely dissolve the sample. The tube was put in the rotor and placed in the open position of the auto sampler and scanned with a BRUKER AVANCE III (400 MHZ).

### 5) HPLC (High-Performance Liquid Chromatography)

HPLC conditions for measuring the solubility of compounds are shown in Table 4 below.

TABLE 4

| Column | C18, 150 [symbol] 4.6 mm, 5 μm | |
|---|---|---|
| Mobile phase A: | 0.05% TFA in H2O | |
| Mobile phase B: | 0.05% TFA in ACN | |
| Gradient: | 0.00 min | 5 % B |
| | 6.50 min | 90% B |
| | 7.00 min | 5% B |
| | 10.00 min | 5% B |

| | |
|---|---|
| Column temperature: | 40° C. |
| Flow rate: | 1.0 ml/minute |
| Detector wavelength: | 220 nm |
| Injection volume: | 5 uL |
| Diluent: | MeOH |

HPLC conditions for measuring the stability of the compounds are shown in Table 5 below.

TABLE 5

| Column | C18, 150 [symbol] 4.6 mm, 5 um | |
|---|---|---|
| Mobile phase A: | 0.05% TFA in H2O | |
| Mobile phase B: | 0.05% TFA in ACN | |
| Gradient: | 0.00 min | 5% B |
| | 20.00 min | 90% B |
| | 25.00 min | 90% B |
| | 25.10 min | 5% B |
| | 30.00 min | 5% B |

| | |
|---|---|
| Column temperature: | 40° C. |
| Flow rate: | 1.0 ml/minute |
| Detector wavelength: | 220 nm |
| Injection volume: | 5 uL |
| Diluent: | MeOH |

### 6) DVS (Dynamic Vapor Sorption)

Using DVS intrinsic manufactured by Surface Measurement Systems Ltd, about 20 mg of the sample was tested for water sorption/desorption profiles at 25° C. under a 0%˜95%˜0% relative humidity (RH) cycle using the following parameters.

Temperature: T=25° C.

Equilibrium: dm/dt: 0.01%/min.

Range of RH (%) measurement step: 0%˜95%˜0%; RH (%) measurement step: 5%

### 7) Preparation of 1-(5-(2-fluorophenyl)-4-methoxy-1-((6-methoxypyridin-3-yl) sulfonyl)-1H-pyrrol-3-yl)-N-methylmethanamine free base

### Step (1): Synthesis of methyl 5-(2-fluorophenyl)-4-methoxy-1-((6-methoxypyridin-3-yl) sulfonyl)-1H-pyrrole-3-carboxylate

Methyl 5-(2-fluorophenyl)-4-methoxy-1H-pyrrole-3-carboxylate (intermediate 1, 1.0 eq., 920 g, 3.69 mol) was dissolved in DMF (9.2 L), and then t-BuOK (2.0 eq., 828 g, 7.38 mmol) was added at 0° C. and stirred for 30 min. 6-Methoxypyridine-3-sulfonyl chloride (1.5 eq., 1.15 kg, 5.54 mol) was added, followed by stirring at 0° C. for 1 hour. Water was added to the reaction solution, and the reaction solution was extracted with ethyl acetate. The organic layer was dried over anhydrous magnesium sulfate, filtered, concentrated, and purified by column chromatography to obtain methyl 5-(2-fluorophenyl)-4-methoxy-1-((6-methoxypyridin-3-yl) sulfonyl)-1H-pyrrole-3-carboxylate as a white solid (1.20 kg, 77.4%).

### Step (2): Synthesis of 5-(2-fluorophenyl)-4-methoxy-1-((6-methoxypyridin-3-yl) sulfonyl)-1H-pyrrol-3-yl) methanol

### Methyl

5-(2-fluorophenyl)-4-methoxy-1-((6-methoxypyridin-3-yl) sulfonyl)-1H-pyrrole-3-carboxylate (1.0 eq., 1.1 kg, 2.62 mol) was dissolved in THF (11.0 mL), and DIBAL 2.0 M in THF solution (3.0 eq., 3.93 L, 7.86 mol) was added dropwise at 0° C., followed by stirring for 30 min. The reaction was completed with a 5% aqueous Rochelle salt solution and the reaction solution was extracted with ethyl acetate. The organic layer was dried over anhydrous magnesium sulfate, filtered, concentrated to obtain 5-(2-fluorophenyl)-4-methoxy-1-((6-methoxypyridin-3-yl) sulfonyl)-1H-pyrrol-3-yl) methanol as a light yellow oil (870 g, 84.8%).

### Step (3): Synthesis of 5-(2-fluorophenyl)-4-methoxy-1-((6-methoxypyridin-3-yl) sulfonyl)-1H-pyrrole-3-carbaldehyde

5-(2-Fluorophenyl)-4-methoxy-1-((6-methoxypyridin-3-yl) sulfonyl)-1H-pyrrol-3-yl) methanol (1.0 eq., 830 g, 2.12 mol) and TEA (4.0 eq., 1.59 kg, 15.7 mol) were dissolved in dimethyl sulfoxide (DMSO) (4.15 L), and then SO₃-pyridine (4.0 eq., 1.35 kg, 8.48 mol) dissolved in DMSO (4.15 L) was added and stirred at room temperature for 1 hour. Water was added to the reaction mixture at 0° C., followed by extraction with ethyl acetate. The organic layer was dried over anhydrous magnesium sulfate, filtered, concentrated to obtain (5-(2-fluorophenyl)-4-methoxy-1-((6-methoxypyridin-3-yl) sulfonyl)-1H-pyrrol-3-carb aldehyde as a yellow solid (722 g, 87.6%).

### Step (4): Synthesis of 1-(5-(2-fluorophenyl)-4-methoxy-1-((6-methoxypyridin-3-yl) sulfonyl)-1H-pyrrole-3-yl)-N-methylmethanamine

5-(2-Fluorophenyl)-4-methoxy-1-((6-methoxypyridin-3-yl) sulfonyl)-1H-pyrrole-3-carbaldehyde (1.0 eq., 715 g, 1.83 mol) was dissolved in methanol (7.2 L), and methylamine in methanol (5.0 eq., 916 g, 9.16 mol) was added. After stirring at room temperature for 1 hour, the reaction product was concentrated, and dissolved in ethanol (7.2 L), and cooled to 0° C. Then, NaBH₄ (2.0 eq., 139 g, 3.66 mol) was added, followed by stirring at room temperature for 1 hour. Water was added to the reaction solution, followed by extraction with ethyl acetate. The organic layer was dried over anhydrous magnesium sulfate, filtered, concentrated, and purified by column chromatography to obtain 1-(5-(2-fluorophenyl)-4-methoxy-1-((6-methoxypyridin-3-yl) sulfonyl)-1H-pyrrole-3-yl)-N-methylmethanamine as a brown oil (430 g, 57.9%).

TABLE 6

| | | HPLC | |
|---|---|---|---|
| | | Retention | LC-MS |
| | | Time | Values |
| Compound Name | NMR Chemical Shift | (min) | [M + H]⁺ |
| 1-(5-(2-fluorophenyl)-4- | ¹H NMR (400 MHZ, CDCl₃) δ | 9.067 | 405 |
| methoxy-1-((6-methoxy- | 8.12 (d, J = 2.4 Hz, 1H), 7.67-7.65 | | |
| pyridin-3-yl)sulfonyl)-1H- | (m, 2H), 7.47-7.43 (m, 1H), 7.25 | | |
| pyrrol-3-yl)-N-methyl- | (dt, J = 7.3, 1.7 Hz, 1H), 7.18 (t, J = | | |
| methanamine | 7.4 Hz, 1H), 7.05 (t, J = 8.8 Hz, | | |
| | 1H), 6.73 (d, J = 8.8 Hz, 1H), 3.99 | | |
| | (s, 3H), 3.94 (s, 2H), 3.46 (s, 3H), | | |
| | 2.64 (s, 3H). | | |

### <Example 1> Preparation of Fumarate Salt of 1-(5-(2-fluorophenyl)-4-methoxy-1-((6-methoxypyridin-3-yl) sulfonyl)-1H-pyrrole-3-yl)-N-methylmethanamine

50.20 mg of 1-(5-(2-fluorophenyl)-4-methoxy-1-((6-methoxypyridin-3-yl) sulfonyl)-1H-pyrrol-3-yl)-N-methylmethanamine free base was weighed and put into a glass vial, and then dissolved in 0.6 mL of isopropyl alcohol at 25° C. Then, 15.13 mg of fumaric acid was added to the vial. The sample was stirred continuously for 24 hours in a magnetic stirrer under room temperature conditions, and then the solid precipitate was separated by centrifugation. Then, the wet solid was further dried under low pressure conditions at room temperature, and the dried solid was analyzed.

The resulting salt was analyzed using XRPD, DSC, TGA and ¹H-NMR.

A brief summary of the results of the analysis above is shown in Table 7 below.

TABLE 7

| | | | ¹H-NMR |
|---|---|---|---|
| | | TGA | (Ratio of |
| | | weight loss | cation and |
| XRPD | DSC | (% w/w) | anion) |
| Crystalline | Melting onset: | Weight loss: | 1:1 |
| Form | 165.97° C. | ~0.0661%, | |
| | Endset: 171.84° C. | 120° C. | |
| | Peak : 169.35° C. | | |
| | (73.07 J/g) | | |

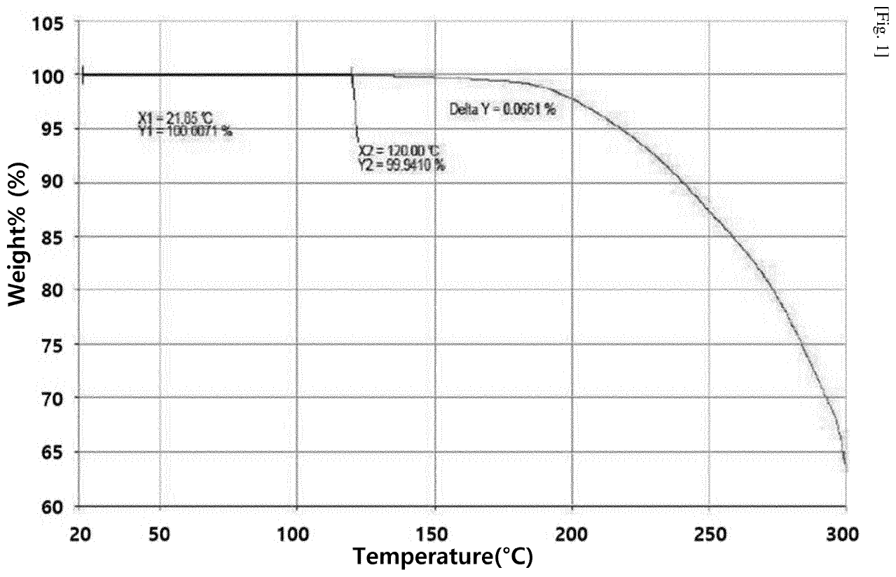

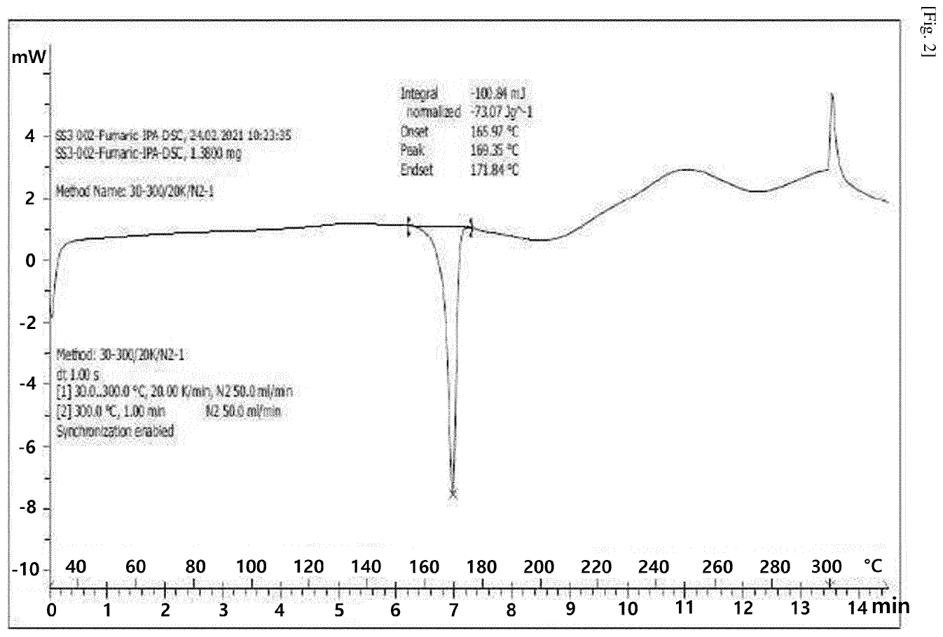

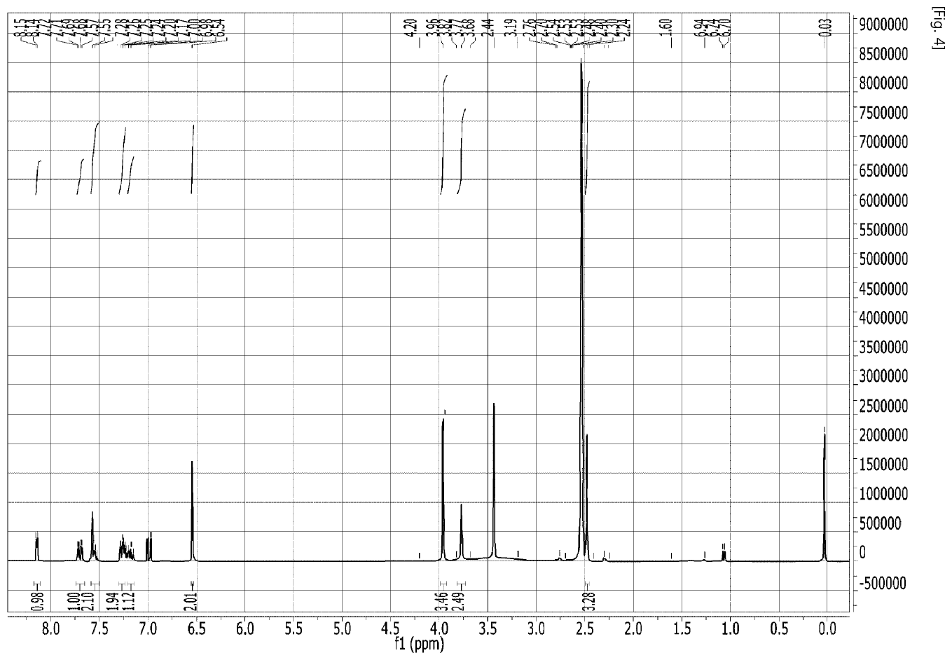

The results of TGA, DSC, XRPD and 1H-NMR analysis are shown in FIGS. 1, 2, 3 and 4, respectively. It was confirmed through the above experimental results that the melting point compared to the free base increased significantly from 57.74° C. to 169.35° C., resulting in excellent stability, and the TGA was lowered to about 0.066% (based on 120° C.) to have an advantage in hygroscopicity, and the drug properties were increased.

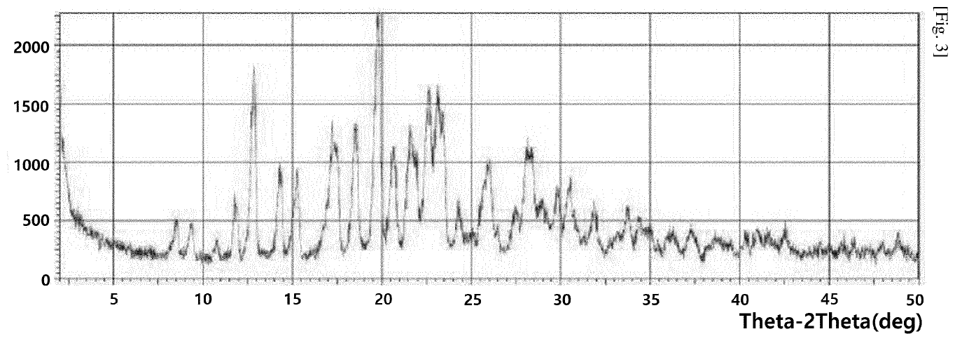

In addition, the prepared compound was measured by XRPD, and as a result, a pattern of 20 values as shown in FIG. 3 was confirmed. In other words, only one crystalline form was confirmed through the above results, and it was confirmed that not only the content of residual solvent was low, but also a melting point (MP) value suitable for commercial use was measured.

### <Example 2> Preparation of Fumarate Salt of 1-(5-(2-fluorophenyl)-4-methoxy-1-((6-methoxypyridin-3-yl) sulfonyl)-1H-pyrrole-3-yl)-N-methylmethanamine

80.06 mg of 1-(5-(2-fluorophenyl)-4-methoxy-1-((6-methoxypyridin-3-yl) sulfonyl)-1H-pyrrol-3-yl)-N-methylmethanamine free base was weighed and put into a glass vial, and then dissolved in 0.4 mL of acetone at 25° C. Then, 24.17 mg of fumaric acid was added to the vial. The sample was continuously stirred for 24 hours in a magnetic stirrer under room temperature conditions, and the solid precipitate was separated by centrifugation and dried at 40° C. for 24 hours.

The resulting salt was analyzed using XRPD, DSC, TGA and ¹H-NMR, and the same results as the crystalline form prepared above under the isopropyl alcohol solvent were obtained.

### <Example 3> Preparation of Fumarate Salt of 1-(5-(2-fluorophenyl)-4-methoxy-1-((6-methoxypyridin-3-yl) sulfonyl)-1H-pyrrole-3-yl)-N-methylmethanamine (Scale-up)

About 500 mg of 1-(5-(2-fluorophenyl)-4-methoxy-1-((6-methoxypyridin-3-yl) sulfonyl)-1H-pyrrol-3-yl)-N-methylmethanamine free base was weighed, put into each glass vial, and dissolved with 3.5 mL of isopropyl alcohol while heating at 25° C. Then, 151.05 mg of fumaric acid was added to the vial. The sample was stirred continuously for 24 hours in a magnetic stirrer at room temperature. After stirring for 24 hours, the solid precipitate was separated by centrifugation and dried at 40° C. for 20 hours. A fumarate salt of 1-(5-(2-fluorophenyl)-4-methoxy-1-((6-methoxypyridin-3-yl) sulfonyl)-1H-pyrrole-3-yl)-N-methylmethanamine (485.95 mg) was obtained as an off-white powder.

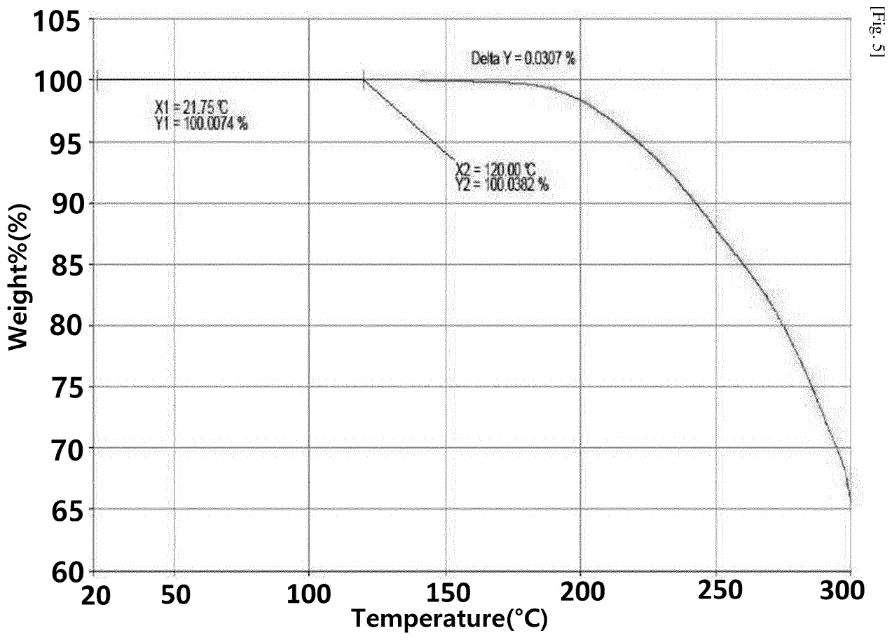

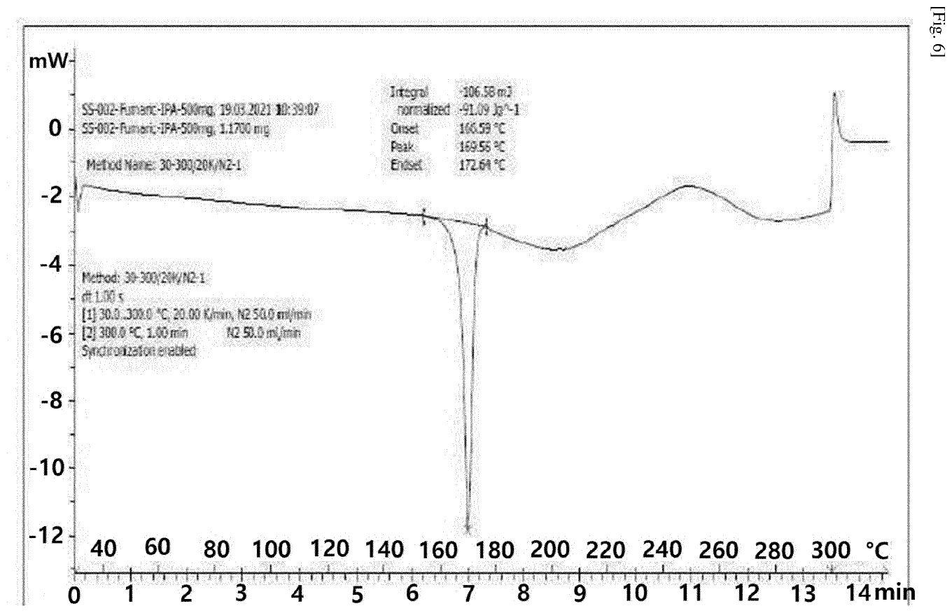

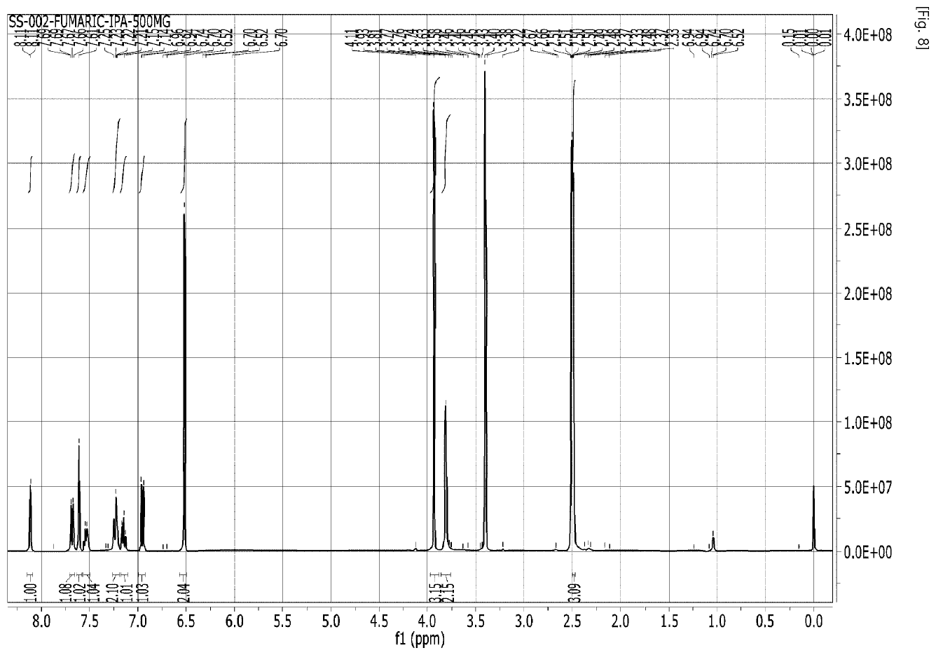

The resulting salt was analyzed using XRPD, DSC, TGA and ¹H-NMR, and the same results as the crystalline form prepared above under the isopropyl alcohol solvent were obtained and shown in FIGS. 5 to 8.

A brief summary of the results of the analysis above is shown in Table 8 below.

TABLE 8

| | | | ¹H-NMR |
|---|---|---|---|
| | | TGA | (Ratio of |
| | | weight loss | cation and |
| XRPD | DSC | (% w/w) | anion) |
| Crystalline | Melting onset: | Weight loss: | 1:1 |
| form | 166.59° C. | ~0.0307%, | |
| | Endset: 172.64° C. | 120° C. | |
| | Peak : 169.56° C. | | |
| | (91.09 J/g) | | |

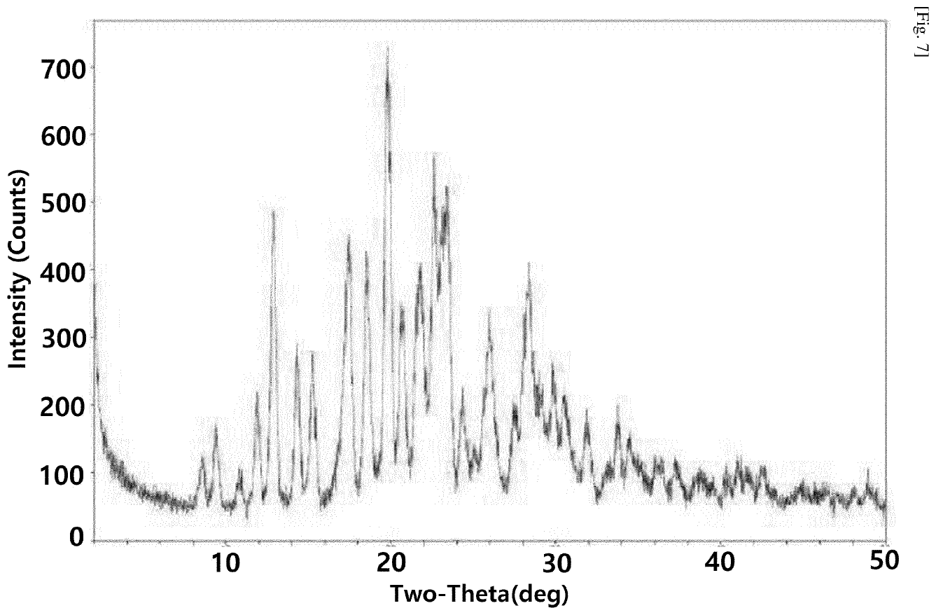

As confirmed above, it was confirmed again that the melting point compared to the free base increased significantly, resulting in a large increase in stability, and the TGA value was also significantly lowered, thus showing very good property even in view of hygroscopicity. Further, as could be confirmed in FIG. 7, XRPD patterns with peaks at 12.87, 14.32, 17.38, 18.55, 19.78, 20.67, 21.74, 22.62, 23.32, 25.95, and 28.27° 2θ±0.2° were shown. In particular, characteristic peaks were identified at 12.87, 17.38, 18.55, 19.78, 22.62, 23.32 and 28.27° 2θ±0.2°

The XRPD pattern confirmed above was confirmed to be the same as the XRPD pattern confirmed in Example 1 above, and thus the formation of one crystalline form could be clearly confirmed again.

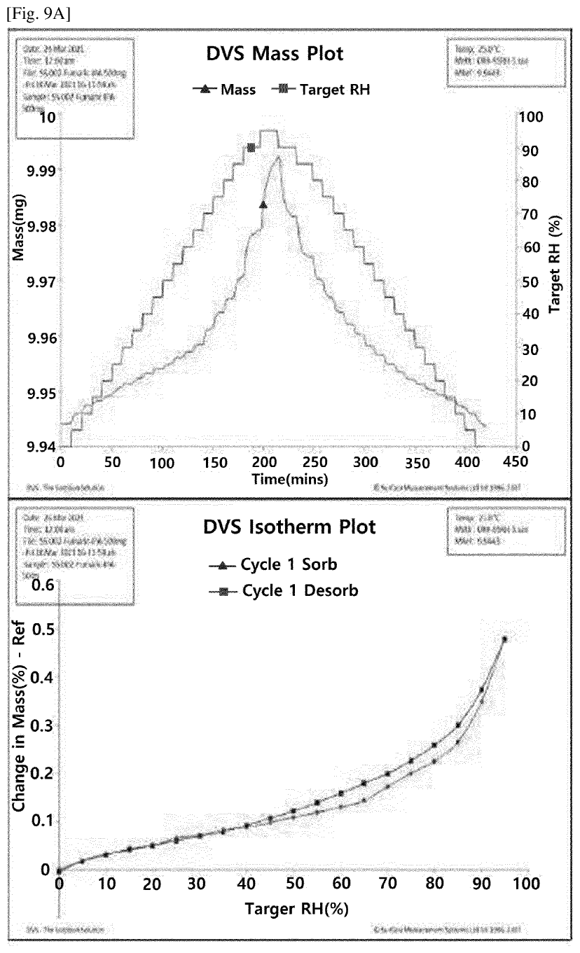

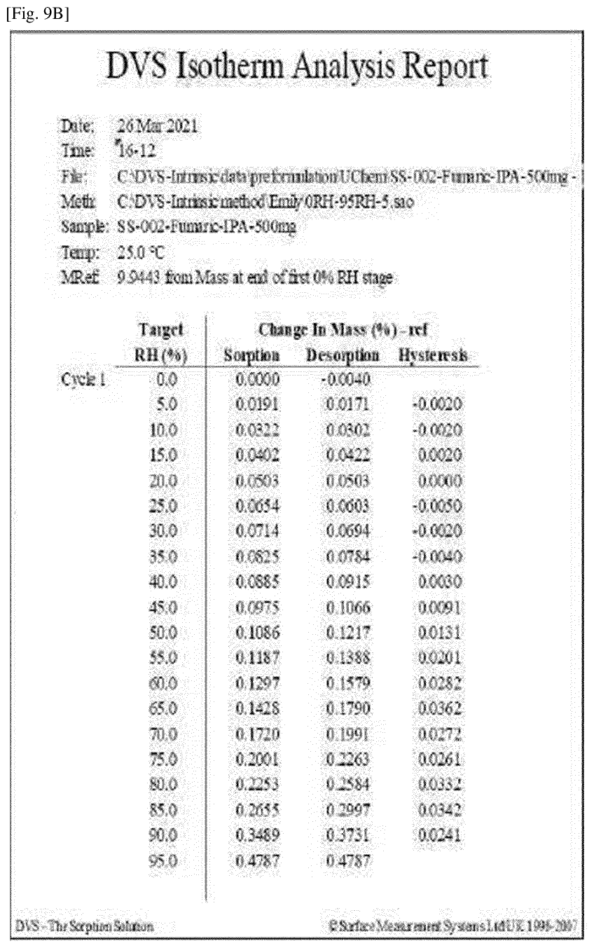

In addition, the DVS values were additionally measured and shown in FIGS. 9A and 9B.

As could be appreciated in FIGS. 9A and 9B, the fumarate salt compound according to the present disclosure exhibited very low hygroscopicity compared to the known hygroscopicity evaluation standard.

### <Comparative Example 1> Preparation of Phosphate Salt of 1-(5-(2-fluorophenyl)-4-methoxy-1-((6-methoxypyridin-3-yl) sulfonyl)-1H-pyrrol-3-yl)-N-methylmethanamine

About 50 mg of 1-(5-(2-fluorophenyl)-4-methoxy-1-((6-methoxypyridin-3-yl) sulfonyl)-1H-pyrrol-3-yl)-N-methylmethanamine free base was weighed and put into a glass vial, and then dissolved in 0.6 mL of isopropyl alcohol at 25° C. Then, 64.74 μL (2 M in MeOH) of phosphoric acid was added to the vial. The sample was stirred continuously for 24 hours in a magnetic stirrer under room temperature conditions, and then the solid precipitate was separated by centrifugation. Then, the wet solid was further dried under low pressure conditions at room temperature, and the dried solid was analyzed.

The resulting salt was analyzed using XRPD, DSC, TGA and ¹H-NMR.

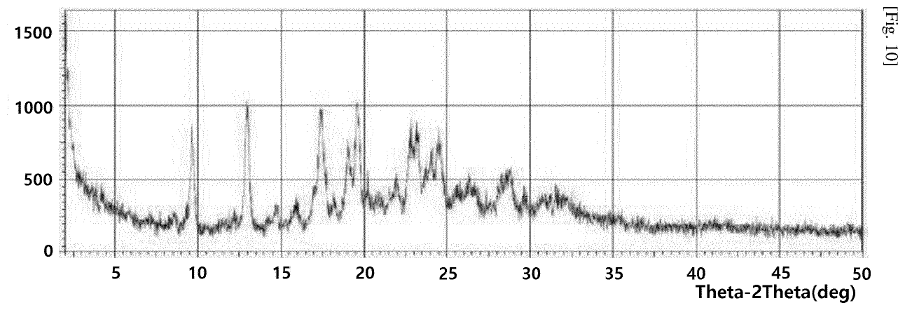

XRPD values are shown in FIG. 10.

In the case of phosphate salt, crystal formation of the salt was confirmed.

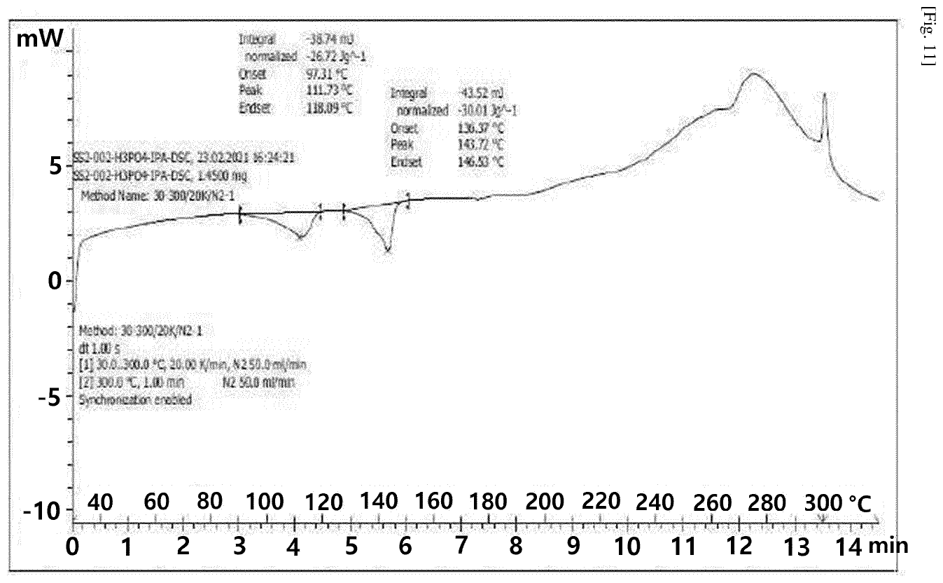

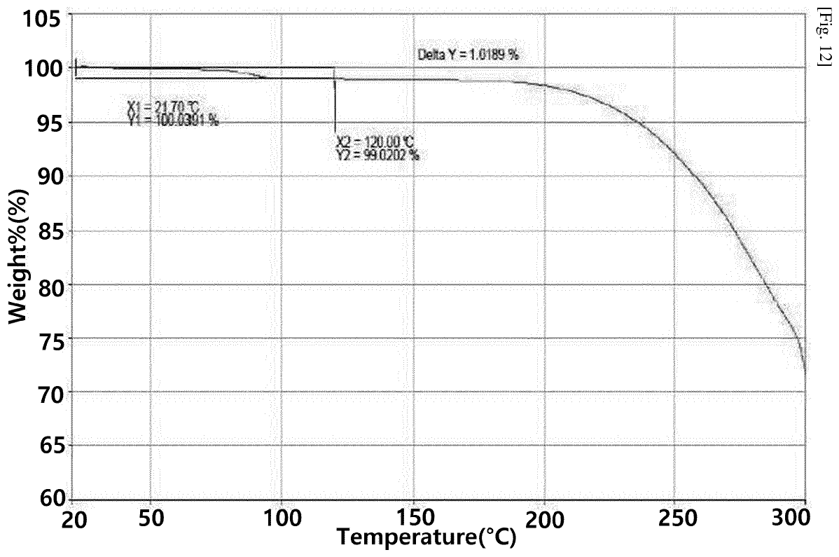

However, DSC analysis confirmed one dehydration/desolvation peak and endothermic transition peak (111.73° C., 143.72° C.) (FIG. 11), and the degree of increase in melting point was lower than that of fumarate salt. Further, it was also confirmed that TGA weight loss was 1.0189%, showing a high hygroscopicity, which was not suitable for being used as a salt (FIG. 12).

### <Comparative Example 2> Preparation of L-Malate Salt of 1-(5-(2-fluorophenyl)-4-methoxy-1-((6-methoxypyridin-3-yl) sulfonyl)-1H-pyrrol-3-yl)-N-methylmethanamine

About 80 mg of 1-(5-(2-fluorophenyl)-4-methoxy-1-((6-methoxypyridin-3-yl) sulfonyl)-1H-pyrrol-3-yl)-N-methylmethanamine free base was weighed and put into a glass vial, and then dissolved in 0.4 mL of acetone while heating at 25° C. Then, 103.59 μL (2 M in MeOH) L-malic acid was added to the vial. The sample was stirred continuously for 24 hours in a magnetic stirrer under room temperature conditions, and then a solvent was evaporated at room temperature.

XRPD for the resulting salt was confirmed. As a result, when L-malic acid was used, the salt had an amorphous form, which confirmed that the availability as a salt was low.

### <Comparative Example 3> Preparation of Citrate Salt of 1-(5-(2-fluorophenyl)-4-methoxy-1-((6-methoxypyridin-3-yl) sulfonyl)-1H-pyrrol-3-yl)-N-methylmethanamine

About 80 mg of 1-(5-(2-fluorophenyl)-4-methoxy-1-((6-methoxypyridin-3-yl) sulfonyl)-1H-pyrrol-3-yl)-N-methylmethanamine free base was weighed and put into a glass vial, and then dissolved in 0.4 mL of acetone while heating at 25° C. Then, 103.59 μL (2 M in MeOH) citric acid was added to the vial. The sample was stirred continuously for 24 hours in a magnetic stirrer under room temperature conditions, and then a solid product was obtained.

XRPD for the resulting salt was confirmed. As a result, when citric acid was used, the salt had an amorphous form, which confirmed that the availability as a salt was low.

### <Comparative Example 4> Preparation of Maleate Salt of 1-(5-(2-fluorophenyl)-4-methoxy-1-((6-methoxypyridin-3-yl) sulfonyl)-1H-pyrrol-3-yl)-N-methylmethanamine

1-(5-(2-Fluorophenyl)-4-methoxy-1-((6-methoxypyridin-3-yl) sulfonyl)-1H-pyrrol-3-yl)-N-methylmethanamine free base was reacted with maleic acid using different solvent conditions such as isopropyl alcohol, acetone, ethyl acetate or ethanol, thereby preparing salts.

As a result of preparing the above salts under different solvent conditions, it was confirmed that when maleic acid was used, no salt of 1-(5-(2-fluorophenyl)-4-methoxy-1-((6-methoxypyridin-3-yl) sulfonyl)-1H-pyrrol-3-yl)-N-methylmethanamine was formed.

### <Comparative Example 5> Preparation of Lactate Salt of 1-(5-(2-fluorophenyl)-4-methoxy-1-((6-methoxypyridin-3-yl) sulfonyl)-1H-pyrrole-3-yl)-N-methylmethanamine

1-(5-(2-Fluorophenyl)-4-methoxy-1-((6-methoxypyridin-3-yl) sulfonyl)-1H-pyrrol-3-yl)-N-methylmethanamine free base was reacted with lactic acid using different solvent conditions such as isopropyl alcohol, acetone, ethyl acetate or ethanol, thereby preparing salts.

As a result of preparing the above salts under different solvent conditions, it was confirmed that when lactic acid was used, no salt of 1-(5-(2-fluorophenyl)-4-methoxy-1-((6-methoxypyridin-3-yl) sulfonyl)-1H-pyrrol-3-yl)-N-methylmethanamine was formed.

### <Experimental Example 1> Evaluation of Solubility in Bio-Relevant Media

Solubility in bio-relevant media, i.e., stimulated gastric fluid (SGF), fasted state simulated intestinal fluid (FaSSIF), and fed state simulated intestinal fluid (FeSSIF) of the free base and the fumarate salt of 1-(5-(2-fluorophenyl)-4-methoxy-1-((6-methoxypyridin-3-yl) sulfonyl)-1H-pyrrole-3-yl)-N-methylmethanamine that was confirmed through the salt screening analysis result, was confirmed.

Buffers for the bio-relevant media were prepared as follows.
Water: Laboratory Milli-Q purified water.
SGF (Stimulated Gastric Fluid): 2.0 g of sodium chloride and 7 mL of hydrochloric acid dissolved in 1000 ml of water was used.
FaSSIF (Fasted State Simulated Intestinal Fluid): A commercial product purchased from Bio-Relevant Company and manufactured according to instructions was used.
FeSSIF (Fed State Simulated Intestinal Fluid): A commercial product purchased from Bio-Relevant Company and prepared according to instructions was used.

For the experiment, about 10 mg of compound (10 mg as free base form) was weighed into each glass vial and then 1 mL of medium was added (final concentration of 10 mg/mL). Then, the sample was continuously stirred in a magnetic stirrer at 37° C. at a speed of 200 rpm. After stirring for 1 hour and 24 hours, 0.5 mL of the sample solution was transferred to a 1.5 mL centrifuge tube and centrifuged at 12,000 rpm for 5 minutes. The supernatant was diluted with suitable methanol and analyzed by HPLC.

The free base was selected as a standard sample to quantify the solubility, and about 10 mg of the free base was weighed into a 25 mL flask and dissolved in methanol to the curve.

The residue of the compound in water was tested by XRPD to determine the solid state.

The experimental results are shown in Table 9 below.

TABLE 9

| | | Solubility-1 h | Solubility-24 h | |
|---|---|---|---|---|
| | | concentration | concentration | |
| Compound | Media | (mg/mL) | (mg/mL) | T (° C.) |
| Free base | water | 8.56 | 8.63 | 37 |
| | SGF | >10 | >10 | 37 |
| | FaSSIF | 1.44 | 1.44 | 37 |
| | FeSSIF | 4.57 | 4.77 | 37 |
| Fumarate salt | water | 6.39 | 5.32 | 37 |
| | SGF | >10 | >10 | 37 |
| | FaSSIF | 7.91 | 7.58 | 37 |
| | FeSSIF | 4.50 | 4.44 | 37 |

As could be confirmed above, the fumarate salt showed excellent solubility in all of SGF, FaSSIF, and FeSSIF, which regulate the pH similar to the in-vivo stomach, small intestine before meals, and small intestine after meals, but in particular exhibited very good solubility under fasted state simulated intestinal fluid (FaSSIF) conditions that mimic the small intestine where most drug absorption occurs. This action shows that the fumarate salt is able to show high solubility regardless of meal, and to have excellent bioavailability, and the like, due to high absorption compared to free base.

### <Experimental Example 2> Drug Stability Evaluation

10 mg and 40 mg of the free base and the fumaric acid salt of 1-(5-(2-fluorophenyl)-4-methoxy-1-((6-methoxypyridin-3-yl) sulfonyl)-1H-pyrrol-3-yl)-N-methylmethanamine were weighed into each vial and stored under stress conditions to investigate stability.

The stress conditions are as follows:
high temperature open room condition: 60° C.;
The bottle neck of the vial was wrapped with aluminum foil with pinholes to avoid contamination, and the samples were analyzed by XRPD and HPLC at the onset (Day 0), week 1 and week 2.

The results thereof are shown in Table 10 below.

TABLE 10

| | Crystalline | | | RT | | | | | | | |
|---|---|---|---|---|---|---|---|---|---|---|---|
| | form | Assay | % | (min) | 6.797 | 7.423 | 7.563 | 9.327 | 10.063 | 10.277 | 10.813 |
| Samples | (XRPD) | % | Impurities | RRT | 0.68 | 0.74 | 0.75 | 0.93 | 1.00 | 1.02 | 1.07 |
| Free | Free form | 100.00 | 0.85 | | | | | 0.18 | 99.15 | | 0.66 |
| base- | Pattern A | | | | | | | | | | |
| Day 0 | | | | | | | | | | | |
| Free | Melted | 67.44 | 20.94 | | 0.43 | 18.86 | 1.00 | 0.18 | 79.06 | 9.95 | 0.57 |
| base at | | | | | | | | | | | |
| 60° C.- | | | | | | | | | | | |
| Week 1 | | | | | | | | | | | |
| Free | Melted | 60.29 | 35.00 | | 0.31 | 13.51 | 4.12 | 0.14 | 65.0 | 16.38 | 0.53 |
| base at | | | | | | | | | | | |
| 60° C.- | | | | | | | | | | | |
| Week 2 | | | | | | | | | | | |
| Fumarate | Crystalline | 100.00 | 0.77 | | | | | 0.17 | 99.23 | | 0.60 |
| salt- | form I | | | | | | | | | | |
| Day 0 | | | | | | | | | | | |
| Fumarate | Crystalline | 105.69 | 0.76 | | | | | 0.17 | 99.24 | | 0.59 |
| salt at | form I | | | | | | | | | | |
| 60° C.- | | | | | | | | | | | |
| Week 1 | | | | | | | | | | | |
| Fumarate | Crystalline | 94.74 | 0.80 | | | | | 0.18 | 99.20 | | 0.62 |
| salt at | form I | | | | | | | | | | |
| 60° C.- | | | | | | | | | | | |
| Week 2 | | | | | | | | | | | |

As could be confirmed in Table 10, the fumarate salt according to the present disclosure showed excellent stability under harsh conditions. On the other hand, it was shown that the free base had a high impurity content under harsh conditions, which had a great limitation in commercial use.

### <Experimental Example 3> Evaluation on Drug Stability

In order to confirm the stability of the fumarate salt of 1-(5-(2-fluorophenyl)-4-methoxy-1-((6-methoxypyridin-3-yl) sulfonyl)-1H-pyrrole-3-yl)-N-methylmethanamine prepared under an isopropyl alcohol solvent, water sorption and/or hydrate formation were confirmed under conditions of 75% RH at 40° C. for 8 weeks.

To proceed with the experiment, 100 mg of the fumarate salt of 1-(5-(2-fluorophenyl)-4-methoxy-1-((6-methoxypyridin-3-yl) sulfonyl)-1H-pyrrole-3-yl)-N-methylmethanamine was put in a vial with a wide mouth (Experimental group 1), and the other sample was put in a vial that is double-packed with a polyethylene bag (Experimental group 2). Then, both samples were placed inside a 40° C., 75% RH chamber and monitored at 2-week intervals.

The XRPD values were checked every two weeks to identify whether or not there was a change in the crystalline form, and the results are shown in Table 11.

TABLE 11

| (Whether or not | | |
|---|---|---|
| XRPD value | Experimental | Experimental |
| changed) | group 1 | group 2 |
| Week 2 | Same | Same |
| Week 2 | Same | Same |
| Week 6 | Same | Same |
| Week 8 | Same | Same |

It could be confirmed from Table 11 above that as compared to the initial XRPD value of the prepared crystalline Form I, no polymorphic change was confirmed and the same XRPD value was shown, and thus the solid phase was stably maintained. In addition, it was confirmed even in the TGA and HPLC analysis conducted at Week 2, Week 4, Week 6, and Week 8 that Experimental group 1 and Experimental group 2 had significantly low hygroscopicity and high purity to have excellent effects in view of drug stability.

1. A fumarate salt of 1-(5-(2-fluorophenyl)-4-methoxy-1-((6-methoxypyridin-3-yl) sulfonyl)-1H-pyrrol-3-yl)-N-methylmethanamine represented by the following Chemical Formula I:

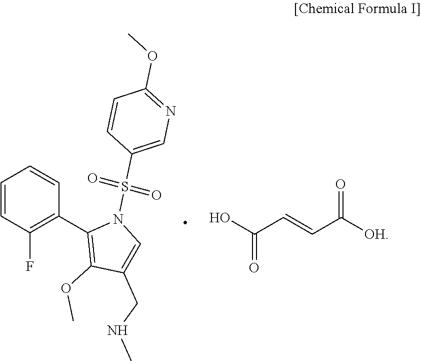 [SMILES: CNCc1cn(S(=O)(=O)c2ccc(OC)nc2)c(-c2ccccc2F)c1OC ; O=C(O)/C=C/C(=O)O]

2. The fumarate salt of claim 1, wherein the fumarate salt has a thermogravimetric analysis (TGA) pattern showing a weight loss of less than 0.1 wt % at 120° C. or less.

3. The fumarate salt of claim 1, wherein the fumarate salt has an endothermic transition peak value at 163 to 175° C. in a differential scanning calorimetry (DSC) graph.

4. The fumarate salt of claim 1, wherein the fumarate salt has an endothermic transition peak value at 169±2° C. in a differential scanning calorimetry (DSC) graph.

5. The fumarate salt of claim 1, wherein the fumarate salt is in a crystalline form.

6. The fumarate salt of claim 5, wherein the crystalline form comprises, in an X-ray powder diffraction (XRPD) graph, at least three diffraction peaks at 2-theta (2θ) angle values selected from the group consisting of 12.87±0.2, 17.38±0.2, 18.55±0.2, 19.78±0.2, 22.62±0.2, 23.32±0.2, and 28.27±0.2.

7. The fumarate salt of claim 6, wherein the crystalline form comprises, in the XRPD graph, diffraction peaks at 2-theta (2θ) angles of 12.87±0.2, 17.38±0.2, 18.55±0.2, 19.78±0.2, 22.62±0.2, 23.32±0.2, and 28.27±0.2.

8. The fumarate salt of claim 5, wherein the crystalline form further comprises, in an X-ray powder diffraction (XRPD) graph, any one or more diffraction peaks at 2-theta (2θ) angle values selected from the group consisting of 14.32±0.2, 20.67±0.2, 21.74±0.2, and 25.95±0.2.

9. (canceled)

10. (canceled)

11. A method for treating gastrointestinal ulcers, gastrointestinal inflammatory diseases or gastric acid-related diseases, comprising: administering to a subject in need thereof a therapeutically effective amount of the fumarate salt according to claim 1.

12. The method of claim 11, wherein the gastrointestinal ulcer, gastrointestinal inflammatory disease or gastric acid-related disease is any one or more selected from the group consisting of peptic ulcer, gastric ulcer, duodenal ulcer, NSAID-induced ulcer, acute stress ulcer, Zollinger-Ellison syndrome, Helicobacter pylori infection, gastritis, erosive esophagitis, non-erosive esophagitis, reflux esophagitis, inflammatory bowel disease, symptomatic gastroesophageal reflux disease (symptomatic GERD), functional dyspepsia, gastric cancer, gastric mucosa-associated lymphoid tissue (MALT) lymphoma, hyperacidity, and upper gastrointestinal bleeding due to invasive stress.

13. (canceled)

14. (canceled)

15. A preparation method of a fumarate salt of 1-(5-(2-fluorophenyl)-4-methoxy-1-((6-methoxypyridin-3-yl) sulfonyl)-1H-pyrrol-3-yl)-N-methylmethanamine represented by the following Chemical Formula I, comprising:
(1) dissolving a compound represented by the following Chemical Formula (II) in a single organic solvent or a mixed solvent to react with fumaric acid;
(2) precipitating a product from a reaction solution obtained in step (1); and
(3) filtering and drying the product of step (2):

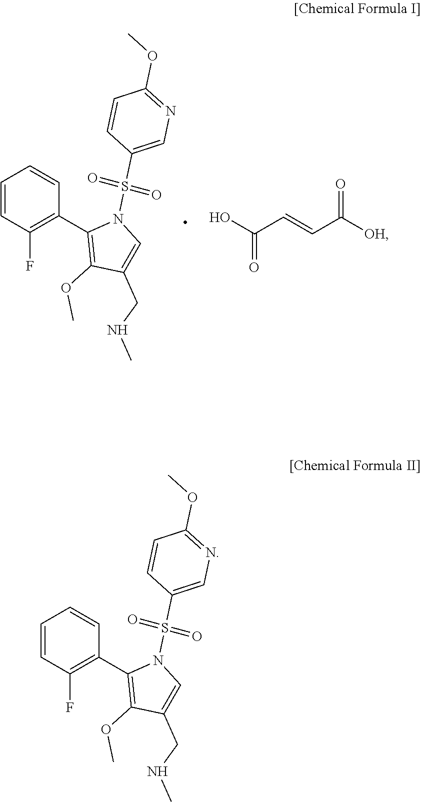 [SMILES: CNCc1cn(S(=O)(=O)c2ccc(OC)nc2)c(-c2ccccc2F)c1OC ; O=C(O)/C=C/C(=O)O ; CNCc1cn(S(=O)(=O)c2ccc(OC)nc2)c(-c2ccccc2F)c1OC]

16. The preparation method of claim 15, wherein the single organic solvent in step (1) is isopropyl alcohol or acetone.

17. The preparation method of claim 15, wherein the step (1) is performed at a temperature of 20 to 40° C.
